(19) United States
(12) Patent Application Publication
(10) Pub. No.: US 2021/0138010 A1
(43) Pub. Date: May 13, 2021

(54) WHOLE GINSENG COMPOSITION USING GINSENG ROOTS, LEAVES AND BERRIES AND METHOD OF PREPARING THE SAME

(71) Applicant: YUHAN CARE CO., LTD., Seoul (KR)

(72) Inventors: Kijun SEONG, Gyeonggi-do (KR); Inae CHUNG, Gyeonggi-do (KR); Ilbum PARK, Gyeonggi-do (KR); Hyunje PARK, Gyeonggi-do (KR); Jongsoo KANG, Seoul (KR)

(21) Appl. No.: 17/041,081

(22) PCT Filed: Mar. 26, 2019
(86) PCT No.: PCT/KR2019/003494

§ 371 Date: Sep. 24, 2020

(30) Foreign Application Priority Data
Mar. 26, 2018 (KR) 10-2018-0034548

(51) Int. Cl. A61K 36/258; A61K 31/704

(52) U.S. Cl. CPC A61K 36/258; A61K 31/704; A61K 2236/37; A61K 2236/53; A61K 2236/331; A61K 2236/333; A61K 2236/51; A61K 2236/31

(57) ABSTRACT

Disclosed are a red ginseng and/or a red ginseng extract obtained by subjecting (raw) ginseng to ultrasonic treatment and ultrahigh-pressure treatment and then steaming and drying the ginseng, and a method of preparing the same. Also disclosed are a whole ginseng composition including the red ginseng extract prepared by the method, a ginseng leaf extract and a ginseng berry extract, and a method of preparing the same. Advantageously, the red ginseng and the red ginseng extract produced by ultrasonic treatment and ultrahigh-pressure treatment according to the present invention have a lower maximum stress than the conventional red ginseng concentrate in terms of physical properties, reduced inconvenience during ingestion and storage, and increased active ingredient contents.

### CROSS-REFERENCE TO RELATED APPLICATIONS

This is a U.S. national phase under the provisions of 35 U.S.C. § 371 of International Patent Application No. PCT/KR19/03494 filed Mar. 26, 2019, which in turn claims priority of Korean Patent Application No. 10-2018-0034548 filed Mar. 26, 2018. The disclosures of such international patent application and Korean priority patent application are hereby incorporated herein by reference in their respective entireties, for all purposes.

### TECHNICAL FIELD

The present invention relates to a red ginseng and/or a red ginseng extract obtained by subjecting (raw) ginseng to ultrasonic treatment and ultrahigh-pressure treatment, and then steaming and drying the ginseng, and a method of preparing the same.

The present invention relates to a whole ginseng composition including the red ginseng extract prepared by the method, a ginseng leaf extract and a ginseng berry extract, and a method of preparing the same.

### BACKGROUND ART

Ginseng (Panax ginseng C.A. Meyer) is a perennial herbaceous plant that belongs to the Panax genus of the Araliaceae family, and has a long history as a medicinal plant all over the world, including the East. Various compounds such as saponin, polysaccharides and phenol compounds are known to be present in ginseng's underground and aboveground parts. Among them, ginsenoside is the most important ingredient, and exhibits a variety of activities including anticancer activity, immune function regulatory activity, anti-inflammatory activity, anti-allergic activity, anti-angiosclerotic activity, anti-stress activity and the like (Lu J M, Yao Q, Chen C. Ginseng compounds: an update on their molecular mechanisms and medical applications. Curr. Vasc. Pharmacol. 2009; 7:293-302.).

At present, about 30 chemical structures of ginsenoside have been found. Major ginsenosides known at present include a total of 34 types of ginsenosides including 13 types of basic ginsenosides and 11 types of transformed (converted) ginsenosides. These ginsenosides are classified into diol-based ginsenosides (protopanaxadiol, PPD) such as ginsenosides Rb1, Rb2, Rc and Rd, and triol-based ginsenosides (protopanaxatriol (PPT)) such as ginsenosides Re, Rg1 and Rf depending on the chemical structure thereof. In particular, ginsenoside Re is a PPT-based ginsenoside, which is known to have various efficacies such as central nervous system inhibitory activity, anti-diabetic activity, activity of secretion acceleration of adrenocorticotropic hormone, and anti-stress activity.

On the other hand, in order to consume the medicinal ingredients of ginseng, ginseng is often processed in the form of medicines or healthy food. Conventionally, to obtain red ginseng, only ginseng roots were used, and ginseng leaves and berries were rarely used. However, leaves and berries of ginseng have a higher saponin content than roots and have a pattern similar to roots, in particular, contain a large amount of ginsenoside Re. Therefore, there is a need for development of a method for utilizing ginsenosides Re.

Meanwhile, Korean Patent No. 1,236,441 discloses a fermented whole ginseng product using a whole ginseng plant and a fermented beverage containing the same, and Korean Patent Laid-Open Publication No. 2016-0028752 discloses a method of processing ginseng berries including ultrahigh-pressure treatment of ginseng berries. However, there is no research on methods for developing food using ginseng leaves or food containing all of ginseng leaves, berries and roots.

On the other hand, a red ginseng concentrate containing 100% raw ginseng-derived raw material is packed at a weight of 100 to 240 g so that it can be consumed at a level of 2 to 6 g a day over 1 to 2 months, and is sold at room temperature and is recommended to be refrigerated after opening. However, when the conventional red ginseng concentrate is stored in the refrigerator to secure safety from microorganisms, the hardness thereof is rapidly increased, which is inconvenient for consumption and use. In addition, the red ginseng concentrate is regulated to contain starch within a level of 1.5% or less. However, as the content of the starch ingredient increases, the red ginseng concentrate hardens during its shelf life and disadvantageously becomes difficult to consume.

Accordingly, the present inventors found that, when the ginseng byproducts of ginseng leaves and berries, which contain large amounts of active ingredients but are not properly used at present, are incorporated into the red ginseng concentrate, the utilization of resources can be enhanced, and that, when the red ginseng concentrate is obtained by physically pre-treating raw ginseng, the amount of active ingredients present can be maximized and problems associated with consumption and storage of conventional red ginseng concentrates caused by the changed physical properties thereof can be solved, thus completing the present invention.

### DISCLOSURE

### Technical Problem

Therefore, it is one object of the present invention to provide a red ginseng and a red ginseng extract, each having improved physical properties and an increased active ingredient content, obtained through physical pretreatment, preferably by ultrasonic treatment and ultrahigh-pressure treatment, of ginseng (raw ginseng), and a method for producing the same. The red ginseng extract produced by the method has lower maximum stress than the conventional red ginseng concentrate in terms of the physical properties thereof, and thus is more convenient to ingest and store.

It is another object of the present invention to provide a whole ginseng composition containing the red ginseng extract, a ginseng leaf extract and a ginseng berry extract, and a method of preparing the same. The present invention provides a whole ginseng composition that can improve the utilization of leaves and berries of ginseng, which are byproducts of ginseng that are not properly used but are discarded at present, and contains reinforced main ginsenosides, unlike conventional red ginseng concentrates.

### Technical Solution

In accordance with the present invention, the above and other objects can be accomplished by the provision of a red ginseng and a red ginseng extract obtained by a method comprising subjecting ginseng (raw ginseng) to ultrasonic treatment and ultrahigh-pressure treatment, and steaming and drying the ginseng (raw ginseng), and a method of preparing the same.

In accordance with another aspect of the present invention, the present invention provides a whole ginseng composition comprising the red ginseng extract, a ginseng leaf extract and a ginseng berry extract. The whole ginseng composition according to the present invention preferably includes 60 to 95% by weight of the red ginseng extract, 3 to 25% by weight of the ginseng leaf extract and 2 to 15% by weight of the ginseng berry extract, but the present invention is not limited thereto.

In accordance with another aspect of the present invention, the present invention provides a method of preparing a whole ginseng composition comprising (a) obtaining red ginseng by subjecting ginseng (raw ginseng) to ultrasonic treatment and ultrahigh-pressure treatment, steaming and drying and then obtaining a red ginseng extract by extracting the red ginseng; (b) obtaining a ginseng leaf extract and a ginseng berry extract by extracting ginseng leaves and ginseng berries, respectively; and (c) mixing the red ginseng extract with the ginseng leaf extract and the ginseng berry extract.

### Advantageous Effects

Advantageously, the red ginseng and the red ginseng extract obtained through ultrasonic treatment and ultrahigh-pressure treatment according to the present invention have a lower maximum stress than conventional red ginseng concentrates in terms of the physical properties thereof, and thus lowered inconvenience during consumption (ingestion) and storage, and have an increased active ingredient content.

In addition, advantageously, the whole ginseng composition containing all of the red ginseng extract, the ginseng leaf extract and the ginseng berry extract according to the present invention has an improved extraction efficiency of ginsenoside, which is a functional substance in red ginseng, due to ultrasonic treatment and ultrahigh-pressure treatment in the process for manufacturing red ginseng, has considerably higher ginsenoside content than that of conventional red ginseng extracts and physical properties superior to conventional products containing only a red ginseng extract, in terms of physical properties, and can utilize ginseng leaves and berries, which are ginseng byproducts rarely used for conventional products.

### BRIEF DESCRIPTION OF THE DRAWINGS

The above and other objects, features and other advantages of the present invention will be more clearly understood from the following detailed description taken in conjunction with the accompanying drawings, in which:

FIGURE 1 is a flowchart illustrating methods of processing ginseng roots, leaves and berries and a process for preparing a whole ginseng composition using a combination of the processing methods according to an embodiment of the present invention.

### DETAILED DESCRIPTION OF THE INVENTION

Unless defined otherwise, all technical and scientific terms used herein have the same meaning as appreciated by those skilled in the field to which the present invention pertains. In general, the nomenclature used herein is well-known in the art and is ordinarily used.

In one aspect, the present invention relates to a red ginseng and a red ginseng extract having improved physical properties and an increased active ingredient, obtained by physical pretreatment of ginseng (raw ginseng), preferably through ultrasonic treatment and ultrahigh-pressure treatment, and a method of preparing the same. In a preferred specific embodiment, the method of preparing red ginseng includes, but is not limited to:

(a) subjecting ginseng (raw ginseng) to ultrasonic treatment and ultrahigh-pressure treatment; and

(b) steaming and drying the ginseng (raw ginseng) to obtain red ginseng.

Further, the method according to the present invention may further include (c) extracting a red ginseng extract from the obtained red ginseng after step (b).

In the present invention, the ginseng or raw ginseng used as a raw material in the preparation of the red ginseng and the red ginseng extract is intended to include roots of ginseng or raw ginseng, and is a portion not containing leaves, berries or stems of ginseng or raw ginseng.

In the method of preparing the red ginseng and red ginseng extract, the ultrasonic treatment and the ultrahigh-pressure treatment in step (a) may be conducted in any order, and may be performed at the same time.

The ultrasonic treatment may be performed under the condition of 10 to 200 kHz, preferably 15 to 150 kHz, and most preferably 20 to 100 kHz, for 5 minutes to 4 hours, preferably 10 minutes to 3 hours, 15 minutes to 2 hours, but the present invention is not limited thereto.

The ultrahigh-pressure treatment may be carried out under the condition of 200 to 800 MPa, preferably 300 to 700 MPa, and most preferably 400 to 600 MPa, for 20 seconds to 10 minutes, preferably 40 seconds to 8 minutes, and most preferably from 1 minute to 6 minutes, but the present invention is not limited thereto.

The step of obtaining red ginseng by steaming and drying in step (b) may be carried out through conventional steaming and drying to obtain red ginseng. For example, the steaming is carried out one or more times at a temperature of 90 to 100° C., preferably 95 to 99° C. for 1 to 5 hours, and more preferably 2 to 3 hours, and the drying is carried out such that the moisture content is 20% or less, preferably 15% or less, in order to obtain red ginseng.

The red ginseng extract in step (c) can be obtained through an extraction method of adding an extraction solvent to the red ginseng obtained in step (b), but the present invention is not limited thereto.

The extraction solvent that can be used to obtain red ginseng extract in the present invention may be water, an organic solvent or a mixture thereof.

Among the extraction solvents, the water may be distilled water or purified water, and the organic solvent may include at least one selected from the group consisting of C₁ to C₆ lower alcohols, acetone, n-hexane, ether, ethyl acetate, diethyl ether, ethyl methyl ketone and chloroform, but the present invention is not limited thereto. The alcohol may be methanol or ethanol. More preferably, the alcohol may be an aqueous solution of 70 to 95 wt % ethanol or methanol. When the ethanol concentration of the aqueous ethanol solution is less than 70%, the active ingredient having physiological activity in ginseng berries or leaves may be extracted in a small amount. When the ethanol concentration thereof is more than 95%, alcohol irritation causes adverse effects on the skin. Thus, an aqueous solution of ethanol is preferably used at the concentration defined above.

Preferably, the solvent for obtaining a red ginseng extract in the present invention may be distilled water, purified water, or a mixture of distilled water and ethanol, but the present invention is not limited thereto.

The extraction of the red ginseng extract may be repeated one or more times, preferably about three times, but the present invention is not limited thereto. The extraction may be carried out by adding an extraction solvent to red ginseng at 70 to 100° C., preferably at 80 to 95° C., and most preferably 85 to 90° C., but the present invention is not limited thereto.

In addition, the method may further include at least one step selected from among centrifugation, filtration, sterilization and concentration steps after step (c) of extracting the red ginseng extraction.

The red ginseng extract prepared by the method according to the present invention is characterized in that ginsenoside, which is a functional substance present in red ginseng, is significantly increased by ultrasonic treatment and the ultrahigh-pressure treatment during the red ginseng manufacturing process, as compared to the conventional ginseng concentrate, and the red ginseng extract has lower maximum stress than a conventional red ginseng concentrate in terms of physical properties, and thus exhibits an effect of easy storage and ingestion.

In another aspect, the present invention provides a whole ginseng composition including all of a red ginseng extract and a ginseng leaf extract, in addition to the red ginseng extract prepared by the method, wherein the present invention is conceived based on the finding that a whole ginseng composition having increased content of an active ingredient derived from ginseng and exhibiting different physical properties from ginseng compositions prepared through conventional methods can be obtained using a mixture of the red ginseng extract with the ginseng leaf extract and the ginseng berry extract.

In the present invention, the whole ginseng composition contains: 60 to 95% by weight, preferably 70 to 91% by weight, and most preferably 75 to 90% by weight of the red ginseng extract; 3 to 25% by weight, preferably 5 to 20% by weight, and most preferably 6 to 18% by weight of a ginseng leaf extract; and 2 to 15% by weight, preferably 3 to 10% by weight, and most preferably 4 to 7% by weight of a ginseng berry extract.

Preferably, the red ginseng extract, the ginseng leaf extract and the ginseng berry extract may be in the form of a concentrated liquid prepared through concentration after extraction, but the present invention is not limited thereto.

In the present invention, the ginseng leaf extract can be obtained by extracting ginseng leaves, and the ginseng berry extract can also be obtained by extracting ginseng berries.

In addition, the method may further include washing and/or steaming ginseng leaves and berries before extraction of the ginseng leaves and berries, and may further include at least one step selected from centrifugation, filtration, sterilization and concentration steps after extraction.

The steaming of the ginseng leaves and berries may be carried out at a temperature of 90 to 100° C., preferably 95 to 99° for 0.5 to 5 hours, preferably 1.5 to 4 hours, and most preferably 2.5 to 200 minutes. This process may be performed at least one time.

The ginseng leaf extract and the ginseng berry extract may be obtained through a method including extracting ginseng leaves and berries by adding an extraction solvent to the ginseng leaves and berries, but the present invention is not limited thereto.

The extraction solvent that can be used for obtaining the ginseng leaf extract and the ginseng berry extract in the present invention may be water, an organic solvent or a mixture thereof.

Among the extraction solvents, water may be distilled water or purified water, and the organic solvent may include at least one selected from the group consisting of C₁ to C6 lower alcohols, acetone, n-hexane, ether, ethyl acetate, diethyl ether, ethyl methyl ketone and chloroform, but the present invention is not limited thereto. The alcohol may be methanol or ethanol. More preferably, the alcohol may be an aqueous solution of a 70 to 95 wt % ethanol or methanol. When the ethanol concentration of the aqueous ethanol solution is less than 70%, the active ingredient having physiological activity in ginseng berries or leaves may be extracted in a small amount. When the ethanol concentration thereof is more than 95%, alcohol irritation causes adverse effects on the skin. Thus, an aqueous solution of ethanol is preferably used at the concentration defined above.

Preferably, the solvent for obtaining the ginseng leaf extract and the ginseng berry extract in the present invention may be distilled water, purified water, or a mixture of distilled water and ethanol, but the present invention is not limited thereto.

In addition, the extraction of the ginseng leaf extract and the ginseng berry extract may be repeated one or more times, preferably about three times, but the present invention is not limited thereto. The extraction may be carried out by adding an extraction solvent to ginseng leaves and ginseng berries at 70 to 100° C., preferably 80 to 98° C., and most preferably 85 to 95° C., but the present invention is not limited thereto.

In the present invention, the whole ginseng composition may further include pharmaceutically or sitologically compatible (available) and acceptable additives. Examples of such additives include, but are not limited to, saccharides, minerals, emulsifiers, organic acids, preservatives, herbal medicinal extracts, and the like.

Examples of useful saccharides include sugars, lactose, maltose, trehalose, sorbitol, corn xylitol, mannitol, fructose, corn syrup solids, honey and the like.

Examples of useful minerals include minerals such as calcium, magnesium, chromium, cobalt, copper, fluoride, germanium, iodine, iron, lithium, magnesium, manganese, molybdenum, phosphorus, potassium, selenium, silicon, sodium, sulfur, vanadium, zinc and the like.

Examples of useful emulsifiers include acacia gum, carboxymethyl cellulose, xanthan gum, pectin and the like.

Examples of useful organic acids include citric acid, malic acid, fumaric acid, adipic acid, phosphoric acid, gluconic acid, tartaric acid, ascorbic acid, acetic acid, phosphoric acid and the like.

Examples of useful preservatives include sodium sorbate, potassium sorbate, calcium benzoate, sodium benzoate, potassium benzoate, EDTA (ethylenediaminetetraacetic acid) and the like.

Examples of useful herbal medicinal extracts include, but are not limited to, herb extracts such as those of ginseng, licorice, Lonicera japonica, Angelica gigas, Aralia cordata, Inula helenium, Saposhnikovia radix, Zizyphus seed, Houttuynia cordata, Forsythiae fructus, Arctium lappa Linne, Epimedium koreanum, Lithospermum root, Sanguisorba officinalis, Cnidii rhizoma, Scrophularia buergeriana, Polygoni cuspidati radix, Platycodon grandiflorum, Aralia elata, Gardenia jasminoides, Phellodendron bark, Phellodendron amurense, Scutellaria baicalensis, Coptis japonica, Rehmannia glutinosa, Paeonia lactiflora, Bupleurum falcatum, Mentha canadensis, Trichosanthes kirilowii Maximowicz, Caragana sinica, Gentianae Macrophyllae radix, Kalpoanax bark, Eucommia ulmoides, Clematis florida, Achyranthes aspera, Acorus gramineus, Cornus officinalis, Schisandra chinensis, Eleutherococcus spp., Rubus coreanus, garlic, Cinnamomum verum, Cuscuta japonica Choisy, Bletilla striata, Angelica dahurica root, Ostericum koreanum Maximowicz, Lindera aggregata, Rheum rhabarbarum, ginger, and frankincense, and encompass all plants. As used herein, the term “extract” means all substances obtained by extracting ingredients contained in natural products, regardless of the extraction method, extraction solvent, extracted ingredient or extract form, and in a broad sense, means all substances obtained by extracting ingredients contained in natural products and subjecting the ingredients to processing or treatment through another method.

In a specific embodiment, the processing or treatment may include additional fermentation or enzymatic treatment of the extract, removal of impurities by centrifugation, filtration or the like, or concentration or drying.

Thus, in this specification, the term “extract” is a broad concept that includes a fermented product, a concentrated product and a dried product. Specifically, in the present specification, the extract may be a liquid-type extract, and is preferably a concentrate (concentrated liquid) of the extract, but the present invention is not limited thereto. In addition, the red ginseng, the ginseng berry extract and the ginseng leaf extract in the present invention are preferably in liquid phases. Thus, the extracted liquid and the extract in the present invention are used interchangeably.

The whole ginseng composition of the present invention can be used as a food, preferably a health functional food. Such food can be formulated into various forms. Examples of such formulations include, but are not limited to, concentrates, sticks, powders, tablets, pills, granules, capsules, suspensions, freeze-drying agents and the like.

The present invention also relates to a method for producing a whole ginseng composition including:

(a) subjecting ginseng (raw ginseng) with ultrasonic treatment and ultrahigh-pressure treatment, steaming and drying the ginseng to obtain red ginseng, and then extracting the red ginseng to obtain a red ginseng extract;

(b) extracting ginseng leaves and ginseng berries to obtain a ginseng leaf extract and a ginseng berry extract, respectively; and

(c) mixing the red ginseng extract with the ginseng leaf extract and the ginseng berry extract.

The method of preparing the whole ginseng composition according to the present invention, the step (a) of obtaining the red ginseng extract and the step (b) of obtaining the ginseng leaf extract and the ginseng berry extract may be carried out at the same time, and the step (b) of obtaining the ginseng leaf extract and the ginseng berry extract may be carried out prior to the step (a) of obtaining the red ginseng extract.

In addition, detailed conditions of steaming and extraction in the step (a) of obtaining the red ginseng extract and the step (b) of obtaining the ginseng leaf extract and the ginseng berry extract may be the same as described above.

Meanwhile, as described above, the method may further include at least one step selected from centrifugation, filtration, sterilization, and concentration steps after the step of extraction for obtaining the red ginseng extract, the ginseng leaf extract and the ginseng berry extract.

The centrifugation may be carried out by a method commonly used in the technical field of the present invention, and may be carried out under appropriate conditions using a conventionally usable centrifuge.

The filtration of the present invention may be carried out by a conventional method after obtaining a supernatant through centrifugation. Non-limiting examples thereof include, but are not limited to, the use of filtration paper or a diatomaceous earth filter. The filtration according to the present invention may be repeated one to five times. In the present invention, the filtrate is made to pass through an ultrafiltration membrane having a molecular weight of 1,000 to 2,000, preferably a molecular weight of 1,000, so that the concentrate (inner liquid) which does not pass through the ultrafiltration membrane is concentrated to 15 to 30%, preferably about 20%. Alternatively, instead of using an ultrafiltration membrane, the extract may be concentrated using a reverse osmosis concentrator (nanofilter system). At this time, the extract may contain ginsenoside Re in a content similar to that realized through a concentration method using ultrafiltration.

In the present invention, the concentration can be carried out through evaporation concentration, freezing concentration, reverse osmosis concentration, or reduced-pressure concentration. The concentration under reduced pressure may be carried out at a pressure of 50 to 100 mmHg pressure at a temperature of 50 to 60° C.

Hereinafter, the present invention will be described in more detail with reference to the following examples. However, it will be obvious to those skilled in the art that these examples are provided only for illustration of the present invention and should not be construed as limiting the scope of the present invention.

### EXAMPLE

### Example 1

### Preparation of Red Ginseng

The rootlets of six-year-old ginseng, purchased from Gimpo Paju Ginseng Nonghyup, were removed, and the ginseng was washed twice with distilled water and treated with ultrasonic waves at 20 to 100 kHz at room temperature for 15 minutes to 1 hour. After the ultrasonic treatment, the ginseng was subjected to ultrahigh-pressure treatment at 400 to 600 MPa for 1 to 6 minutes. Then, the red ginseng was steamed at 95 to 99° C. for 2 to 3 hours and then dried to 15% moisture content. For analysis of the main ginsenoside ingredients of raw ginseng and red ginseng, ginsengs of Control and Test groups were ground, 1 g of each group was added to 70 ml of methanol, and the mixture was extracted under reflux for 1 hour and then concentrated under reduced pressure. After dissolving in acetonitrile, the resulting solution was filtered, and Test and Control groups classified as shown in Table 1 were quantitatively analyzed using HPLC.

As shown in Table 2, when the ginseng was subjected either to ultrasonic treatment or to ultrahigh-pressure treatment, there was no significant difference in the total amount of ginsenosides between the treatment group and the non-treatment group (control group). However, when the ginseng was subjected to both the ultrasonic treatment and the ultrahigh-pressure treatment, the treatment group exhibited higher ingredient contents than the control group. In this example, the ultrahigh-pressure treatment was performed after the ultrasonic treatment, but the present invention is not limited to this treatment order.

TABLE 1
Setting of ultrasonic treatment and ultrahigh-pressure treatment groups

| | | Process of preparing red ginseng | |
|---|---|---|---|
| Process | Raw ginseng | (raw ginseng→ red ginseng) | Red ginseng |
| Non-treatment | Control group 1 | Steaming → drying | Control group 2 |
| Ultrasonic treatment | Test group 1 | | Test group 4 |
| Ultrahigh-pressure treatment | Test group 2 | | Test group 5 |
| Ultrasonic treatment + | Test group 3 | | Test group 6 |
| ultrahigh-pressure treatment | | | |

TABLE 2
Comparison of ginsenoside content (mg/g) upon ultrasonic treatment and ultrahigh-pressure treatment

| | Raw ginseng | | | | Red ginseng | | | |
|---|---|---|---|---|---|---|---|---|
| | | | | Test group 3 | | | | Test group 6 |
| | | | | (Ultrasonic | | | | (Ultrasonic |
| | | | Test group 2 | treatment + | | | Test group 5 | treatment + |
| | | Test group 1 | (ultrahigh- | ultrahigh- | | Test group 4 | (ultrahigh- | ultrahigh- |
| Compound | Control group 1 | (Ultrasonic | pressure | pressure | Control group 2 | (Ultrasonic | pressure | pressure |
| Name | (Non-treatment) | treatment) | treatment) | treatment) | (Non-treatment) | treatment) | treatment) | treatment) |
| G.Rg1 | 1.508 | 0.838 | 0.794 | 1.173 | 1.183 | 1.115 | 1.691 | 2.049 |
| G.Re | 0.640 | 1.164 | 1.470 | 1.759 | 1.708 | 1.429 | 1.268 | 3.214 |
| G.Rf | 0.473 | 0.307 | 0.271 | 0.394 | 1.499 | 1.272 | 1.597 | 2.385 |
| G.Rh1 | 0.000 | 0.000 | 0.102 | 0.081 | 1.527 | 1.358 | 1.341 | 1.820 |
| G.F3 | 0.000 | 0.183 | 0.000 | 0.000 | 0.590 | 0.632 | 1.061 | 1.511 |
| G.Rb1 | 2.085 | 1.756 | 2.705 | 3.337 | 7.944 | 7.569 | 9.547 | 11.213 |
| G.Rc | 0.622 | 0.568 | 0.952 | 1.178 | 2.100 | 2.477 | 1.780 | 1.983 |
| G.Rb2 | 0.547 | 0.336 | 0.797 | 0.980 | 1.953 | 1.994 | 0.984 | 1.197 |
| G.F1 | 0.000 | 0.000 | 0.000 | 0.000 | 0.000 | 0.000 | 0.000 | 0.000 |
| G.Rb3 | 0.000 | 0.000 | 0.058 | 0.078 | 0.129 | 0.145 | 0.088 | 0.104 |
| G.Rd | 0.049 | 0.061 | 0.054 | 0.130 | 0.383 | 0.406 | 0.372 | 0.289 |
| G.F2 | 0.000 | 0.000 | 0.000 | 0.000 | 0.000 | 0.000 | 0.000 | 0.000 |
| G.Rg3(S) | 0.000 | 0.000 | 0.000 | 0.000 | 0.837 | 0.859 | 0.912 | 0.907 |
| G.Rg3(R) | 0.000 | 0.000 | 0.000 | 0.000 | 0.653 | 0.706 | 0.728 | 0.709 |
| Compound K | 0.000 | 0.000 | 0.000 | 0.000 | 0.212 | 0.261 | 0.204 | 0.229 |
| G.Rk1 | 0.000 | 0.000 | 0.000 | 0.000 | 4.632 | 4.302 | 4.362 | 4.186 |
| G.Rg5 | 0.000 | 0.000 | 0.000 | 0.000 | 0.291 | 0.322 | 0.347 | 0.348 |
| G.Rh2(S) | 0.000 | 0.000 | 0.000 | 0.000 | 0.000 | 0.000 | 0.000 | 0.000 |
| Protopanaxadiol | 0.133 | 0.146 | 0.527 | 0.912 | 0.000 | 0.000 | 0.000 | 0.231 |
| G.F11 | 1.149 | 1.947 | 1.444 | 2.383 | 0.000 | 1.320 | 0.948 | 0.000 |
| Total | 7.21 | 7.31 | 9.17 | 12.40 | 59.84 | 69.19 | 63.93 | 74.59 |
| Total of | 3.59 | 2.59 | 3.50 | 4.51 | 10.62 | 10.25 | 12.88 | 14.88 |
| G.Rg1, Rb1 and Rg3 | | | | | | | | |

### Example 2

### Preparation of Whole Ginseng Composition

Distilled water was added to the red ginseng obtained in Example 1, and the mixture was extracted at 85 to 90° C., followed by centrifugation and filtration to remove floating matter. The residue was concentrated again to obtain a dark brown red ginseng concentrate. Separately, ginseng leaves and berries were washed, steamed at 95 to 99° C. for 150 to 200 minutes and then extracted at 85 to 95° C. using distilled water, and the floating matter was removed by centrifugation and filtration and then concentrated to obtain a ginseng leaf concentrate and a ginseng berry concentrate. 76 to 89 wt % of the red ginseng concentrate, 5 to 20 wt % of the ginseng leaf concentrate and 1 to 10 wt % of the ginseng berry concentrate were mixed and concentrated to obtain a whole ginseng concentrate.

In order to analyze main ginsenoside ingredients in the red ginseng concentrate, the ginseng leaf concentrate and the ginseng berry concentrate, 1 g of the red ginseng concentrate, 1 g of the ginseng leaf concentrate and 1 g of the ginseng berry concentrate were added to 70 ml of methanol, and the mixture was extracted under reflux for 1 hour and then concentrated under reduced pressure. The resulting concentrate was dissolved in acetonitrile, and the solution was filtered and quantitatively analyzed using HPLC. For analysis of the total polyphenol content, 1 g of the red ginseng concentrate, 1 g of the ginseng leaf concentrate and 1 g of the ginseng berry concentrate were added to 50 ml of distilled water, extracted under ultrasonication for 30 minutes and diluted with distilled water, and the resulting solution was mixed with Folin-Denis reagent and 35% sodium carbonate. The resulting mixture was allowed to stand in the dark for 30 minutes and was quantitatively analyzed by measurement of absorbance at 760 nm. In addition, for analysis of total flavonoid ingredients, 1 g of the red ginseng concentrate, 1 g of the ginseng leaf concentrate and 1 g of the ginseng berry concentrate were dissolved in 20 ml of 90% ethanol and centrifuged (3,000 rpm, 10 minutes) to obtain the supernatant. The supernatant was extracted three times with 80% ethanol. 1.5 mL of ethanol, 0.1 mL of a 10% aluminum nitrate solution, 0.1 mL of a 1M potassium acetate solution and 2.8 mL of distilled water were added to 50 mL of the extract, the resulting mixture was allowed to stand for 40 minutes, and the absorbance of the reaction solution was measured at 415 nm.

As shown in Table 3, the total ginsenoside content in the ginseng berry concentrate was about 3 times that of the red ginseng concentrate using only ginseng roots. It could be seen that, among them, ginsenoside Re, Rg2 and Rd ingredients were present in higher amounts in the ginseng berry concentrate.

In addition, Table 4 shows analysis results of the total polyphenol and total flavonoid of leaf, berry and root concentrates. Regarding total polyphenol and total flavonoid contents per unit solids (1 Bx), the leaf and berry concentrates contain more total polyphenols and total flavonoids than root concentrates. In addition, as can be seen from Table 5, the whole ginseng concentrate, prepared using all of the root, leaf and berry concentrates, contained higher main ginsenoside ingredient contents (including total ginsenoside content) than those of general ginseng concentrates prepared using only ginseng roots.

TABLE 3
Comparison of ginsenoside content in ginseng berry, root and leaf concentrates (mg/g)

| | | | | | | | | | | | | | Total |
|---|---|---|---|---|---|---|---|---|---|---|---|---|---|
| | Rg1 | Re | Rf | Rh1 | Rg2 | Rb1 | Rc | Rb2 | Rb3 | Rd | Rg3 | Rh2 | ginsenoside |
| Red | 1.31 | 1.95 | 1.41 | 1.69 | 1.78 | 3.91 | 1.81 | 3.45 | 0.41 | 1.22 | 3.92 | | 22.86 |
| ginseng | | | | | | | | | | | | | |
| concentrate | | | | | | | | | | | | | |
| (root) | | | | | | | | | | | | | |
| Ginseng | 2.55 | 15.32 | 0.41 | 1.63 | 7.34 | 4.64 | 6.99 | 12.54 | 1.11 | 5.71 | 5.97 | 0.00 | 64.21 |
| berry | | | | | | | | | | | | | |
| concentrate | | | | | | | | | | | | | |
| Ginseng | 0.03 | 0.06 | 0.14 | 2.47 | 3.22 | 0.02 | 0.08 | 0.16 | 0.00 | 1.87 | 2.62 | 3.62 | 14.28 |
| leaf | | | | | | | | | | | | | |
| concentrate | | | | | | | | | | | | | |

TABLE 4
Comparison of total polyphenol and total flavonoid
contents according to ginseng part

| Analysis | Ingredient contents per unit solids (mg/solids (1Bx) | |
|---|---|---|
| items | Total polyphenol | Total flavonoid |
| Leaf concentrate | 0.338 | 0.039 |
| Berry concentrate | 0.334 | 0.013 |
| Root concentrate | 0.188 | 0.003 |

TABLE 5
Comparison in ginsenoside content (mg) between general red ginseng concentrate (100 g) and whole ginseng concentrate (100 g)

| | Rg1 | Re | Rf | Rh1 | Rg2 | Rb1 | Rc | Rb2 | Rb3 | Rd | Rg3 | Rh2 | Total |
|---|---|---|---|---|---|---|---|---|---|---|---|---|---|
| General | 142.27 | 212.16 | 153.06 | 183.41 | 192.82 | 423.97 | 196.65 | 374.68 | 43.98 | 131.97 | 425.50 | 0.00 | 2480.48 |
| ginseng | | | | | | | | | | | | | |
| concentrate | | | | | | | | | | | | | |
| Whole | 135.14 | 239.23 | 138.78 | 190.01 | 223.43 | 392.62 | 198.45 | 375.66 | 42.78 | 152.29 | 420.85 | 31.46 | 2540.69 |
| ginseng | | | | | | | | | | | | | |
| concentrate | | | | | | | | | | | | | |

### Example 3

### Comparison of Physical Properties of Whole Ginseng Concentrate

Texture profiles of the whole ginseng concentrate obtained in Example above and other ginseng concentrates prepared by extracting and concentrating only red ginseng without mixing other additives (red ginseng concentrate 1: red ginseng concentrate prepared using the same preparation apparatus and raw ginseng derived from the same area/red ginseng concentrate 2: general red ginseng concentrate sold well were analyzed using a rheometer (Sun Scientific, CR-100, No 2 Adapter). Samples stored at room temperature and under refrigeration for one day or longer were measured, respectively, and the results are shown in Tables 6 and 7.

TABLE 6
Comparison in texture profiles between whole ginseng concentrate and other red ginseng concentrates stored at room temperature

| | | | Maximum | Maximum | | | | | |
|---|---|---|---|---|---|---|---|---|---|
| | | | stress | stress | | | | | |
| | | Storage | (Max1) | (Max2) | Springiness | Cohesiveness | Gumminess | Brittleness | Adhesiveness |
| No | Name | temperature | g | g | % | % | G | g | G |
| 1 | Whole | Room | 11.0 | 9.0 | 119.2 | 100.8 | 11.1 | 13.2 | −6.0 |
| | ginseng | temperature | | | | | | | |
| | concentrate | | | | | | | | |
| 2 | Red ginseng | Room | 26.0 | 26.0 | 101.2 | 88.6 | 23.0 | 23.3 | −17.0 |
| | Concentrate 1 | temperature | | | | | | | |
| | (same preparation | | | | | | | | |
| | apparatus) | | | | | | | | |
| 3 | Red ginseng | Room | 21.0 | 18.0 | 115.3 | 98.7 | 20.7 | 23.9 | −11.0 |
| | Concentrate 2 | temperature | | | | | | | |
| | (general) | | | | | | | | |

TABLE 7
Comparison in texture profiles between whole ginseng concentrate and other red ginseng concentrates stored in refrigerator

| | | | Maximum | Maximum | | | | | |
|---|---|---|---|---|---|---|---|---|---|
| | | | stress | stress | | | | | |
| | | Storage | (Max1) | (Max2) | Springiness | Cohesiveness | Gumminess | Brittleness | Adhesiveness |
| No | Name | temperature | g | g | % | % | g | g | G |
| 1 | Whole | Refrigerated | 28.0 | 29.0 | 105.6 | 89.4 | 25.0 | 26.4 | −19.0 |
| | ginseng | | | | | | | | |
| | concentrate | | | | | | | | |
| 2 | Red ginseng | Refrigerated | 46.0 | 51.0 | 101.9 | 86.5 | 39.8 | 40.5 | −38.0 |
| | concentrate 1 | | | | | | | | |
| | (same preparation | | | | | | | | |
| | apparatus | | | | | | | | |
| 3 | red ginseng | Refrigerated | 37.0 | 30.0 | 102.9 | 85.6 | 31.7 | 32.6 | −20.0 |
| | concentrate 2 | | | | | | | | |
| | (general) | | | | | | | | |

As can be seen from Tables 6 and 7, the whole ginseng concentrate had the lowest maximum stress values (Max1) both at room temperature and in the refrigerated state, and thus had the softest texture both at room temperature and in the refrigerated state. Also, it can be seen that springiness and cohesiveness of the whole ginseng concentrate were the highest, and the absolute values of gumminess, brittleness and adhesiveness were the lowest. These results indicate that the whole ginseng concentrate has higher springiness and better resilience, and thus sticks less to the teeth during consumption than general red ginseng concentrates.

Regarding such a whole ginseng concentrate, it is considered that the starch particles, which are usually contained in an amount less than 1.5% in red ginseng concentrates, are gelatinized through combined physical treatment of ultrasonic treatment and ultrahigh-pressure treatment, and thus provide properties different from general red ginseng concentrates. In particular, red ginseng concentrate products will be consumed over a period of one to two months and are usually required to be refrigerated after opening so that they should be easy to consume (digest) even in the refrigerated condition. The whole ginseng concentrate showed the maximum stress of other ginseng products at room temperature even in the refrigerated state, which indicates that the ease of ingestion is superior to that of the general red ginseng concentrate. In addition, the red ginseng concentrate hardens during long-term storage and undergoes deterioration in quality. The raw ginseng concentrate has a low maximum stress value even from the beginning and thus hardens less than general red ginseng concentrate even during long-term storage.

Although specific configurations of the present invention have been described in detail, those skilled in the art will appreciate that this description is provided as preferred embodiments for illustrative purposes and should not be construed as limiting the scope of the present invention. Therefore, the substantial scope of the present invention is defined by the accompanying claims and equivalents thereto.

1. A method of preparing red ginseng comprising:
(a) subjecting ginseng (raw ginseng) to ultrasonic treatment and ultrahigh-pressure treatment; and
(b) obtaining red ginseng by steaming and drying the ginseng.

2. The method of preparing red ginseng of claim 1, wherein the ultrasonic treatment in step (a) is carried out under the condition of 10 to 200 kHz for 5 minutes to 4 hours and the ultrahigh-pressure treatment in step (a) is carried out under the condition of 200 to 800 MPa for 20 seconds to 10 minutes.

3. The method of preparing red ginseng of claim 1, wherein the steaming in step (b) is carried out at a temperature of 90 to 100° C. for 1 to 5 hours and is repeated one or more times.

4.-9. (canceled)

10. A whole ginseng composition comprising the red ginseng extract prepared by the method of claim 1, a ginseng leaf extract and a ginseng berry extract.

11. The whole ginseng composition of claim 10, comprising 60 to 95% by weight of the red ginseng extract, 3 to 25% by weight of the ginseng leaf extract and 2 to 15% by weight of the ginseng berry extract.

12. The whole ginseng composition of claim 10, wherein the ginseng leaf extract and the ginseng berry extract are obtained by a method comprising adding an extraction solvent to ginseng leaves and berries and extracting the ginseng leaves and berries.

13. The whole ginseng composition of claim 12, wherein the extraction solvent used to obtain the ginseng leaf extract and the ginseng berry extract is water, an organic solvent or a mixture thereof.

14. The whole ginseng composition of claim 12, comprising adding the extraction solvent to ginseng leaves and berries and extracting the ginseng leaves and berries at a temperature of 70 to 100° C.

15. The whole ginseng composition of claim 12, wherein the ginseng leaf extract and the ginseng berry extract are obtained by a method further comprising washing and/or steaming the ginseng leaves and berries before the extraction using the extraction solvent.

16. The whole ginseng composition of claim 12, wherein the ginseng leaf extract and the ginseng berry extract are obtained by a method further comprising at least one step selected from centrifugation, filtration, sterilization and concentration steps after the extraction using the extraction solvent.

17. The whole ginseng composition of claim 12, wherein the red ginseng extract, the ginseng leaf extract and the ginseng berry extract are in the form of concentrates obtained by concentration after extraction.

18.-19. (canceled)

20. A method of preparing the whole ginseng composition of claim 10, comprising:
(a) obtaining red ginseng by subjecting ginseng (raw ginseng) to ultrasonic treatment and ultrahigh-pressure treatment, steaming and drying and then obtaining a red ginseng extract by extracting the red ginseng;
(b) obtaining a ginseng leaf extract and a ginseng berry extract by extracting ginseng leaves and ginseng berries, respectively; and
(c) mixing the red ginseng extract with the ginseng leaf extract and the ginseng berry extract.
